(19) United States
(12) Patent Application Publication
(10) Pub. No.: US 2023/0285277 A1
(43) Pub. Date: Sep. 14, 2023

(54) MULTI-LAYERED HYDROGEL DEVICES FOR INTRAVAGINAL APPLICATIONS

(71) Applicant: AQUAFIT INTIMATE LTD., Tel Aviv (IL)

(72) Inventors: Rebecca STERNBERG, Tel Aviv (IL); Vered ITALIANO, Kfar Ben Non (IL); Varda MESSER, Tel Aviv (IL)

(21) Appl. No.: 18/016,494

(22) PCT Filed: Jul. 14, 2021
(86) PCT No.: PCT/IL2021/050859

§ 371 Date: Jan. 17, 2023

Related U.S. Application Data
(60) Provisional application No. 63/053,684, filed on Jul. 19, 2020.

(51) Int. Cl. A61K 9/00; A61K 47/36; A61L 31/14

(52) U.S. Cl. CPC A61K 9/0036; A61K 47/36; A61L 31/145

(57) ABSTRACT

Provided herein are an intravaginal delivery device comprising a plurality of distinct hydrogel layers, and methods of using same for treating a disease or disorder.

### FIELD OF THE INVENTION

Provided herein are an intravaginal delivery device comprising a plurality of distinct hydrogel layers, methods for the preparation thereof and methods of using same for vaginal care.

### BACKGROUND

Intravaginal applicators may be used for delivery of nutrients and pharmaceuticals in order to provide topical and/or systemic impact. Hydrogels are three-dimensional, hydrophilic, polymeric networks, with chemical or physical cross-links, capable of imbibing and maintaining large volumes of water or biological fluids.

Hydrogel articles for intravaginal application are disclosed, for example, in WO 2019/175864 and U.S. Pat. Nos. 3,867,329; 4,480,642; 4,480,642; and 4,977,904. Cool Water Cones is a commercial dilator device made of gel containing more than 90% water.

There is an unmet need for reusable, sophisticated intravaginal devices capable of exerting several effects, simultaneously or sequentially, through multiple hydrogel layers, and configured for smart controlled delivery of beneficial substances to the vaginal environment for the purpose of vaginal care and therapy.

### SUMMARY

There is provided an intravaginal delivery device having a plurality of hydrogel layers, wherein adjacent hydrogel layer layers are attached to one another, and wherein the layers of the plurality of hydrogel layers are different from one another by content.

The term “hydrogel layer” as used herein refers to a 3-D structure, particularly, a substantially solid 3-D mesh or network of polymers. Thus, a hydrogel layer is not limited to a flat hydrogel sheet, but rather may have any desired 3-D shape, as further disclosed and illustrated herein.

Advantageously, the intravaginal delivery device disclosed herein is capable of producing multiple effects related to vaginal care and/or therapy, afforded by a plurality of hydrogel layers that form together a homogenous elongated structure, yet, each layer is designed to exert a different effect. Furthermore, the intravaginal delivery device can deliver a plurality of active agents which cannot be combined in the same composition, such as, hydrophilic and hydrophobic active ingredients or active agents that when combined interfere with, or inhibit, the activity of one another. In addition, the intravaginal delivery device is useful for delivery of active agents under various controlled release regimens, wherein the timing of release may be activated by vaginal triggers, such as, pH, temperature, and by the geometry (i.e. multilayered arrangement) of the device.

Surprisingly, despite the sophisticate multi-layered structure, the device is stable and maintains its structure during use, and moreover during multiple uses, as adjacent layers well adhere to one another. The strong and reliable attachment between neighboring hydrogel layers is achieved during the preparation process, through sequential cooling, or by pressing pre-gelled layers.

The advanced multi-layer structure disclosed herein can be reused multiple times, without compromising on its therapeutic impact. Moreover, the light weight, elongated structure, as well as smooth and humid surface, render the multi-layered device easy and convenient to grip and to slide into the vaginal canal.

There is provided, in some embodiments, an intravaginal delivery device comprising a plurality of hydrogel layers, wherein adjacent hydrogel layer layers are attached to one another, and wherein the layers of the plurality of hydrogel layers are different from one another by content.

In some embodiments, the plurality of hydrogel layers is arranged concentrically and comprises a core hydrogel layer and an external hydrogel layer.

In some embodiments, the intravaginal delivery device further comprises at least one transitional hydrogel layer.

In some embodiments, the plurality of hydrogel layers is arranged in a stack.

In some embodiments, at least one layer of the plurality of layers is a foundational layer comprising at least one other layer dispersed therewithin.

In some embodiments, the at least one other layer is in the form of particles.

In some embodiments, the at least one other layer comprises a plurality of distinct layers.

In some embodiments, the particles are randomly dispersed within the foundational layer.

In some embodiments, at least one layer of the plurality of hydrogel layers comprises a hybrid hydrogel.

In some embodiments, at least one layer of the plurality of hydrogel layers comprises a pharmaceutical composition comprising at least one active agent.

In some embodiments, there is provided a kit comprising the intravaginal delivery device disclosed herein, a cover configured to enclose the intravaginal delivery device therewithin, and instructions for use of the intravaginal delivery device for delivering, intravaginally, a plurality of active agents.

In some embodiments, the cover is a solid cover.

In some embodiments, the cover is configured to hermetically enclose the intravaginal delivery device therewithin.

In some embodiments, the kit further comprises a pH indicator.

In some embodiments, there is provided a method for intravaginal delivery of a plurality of active agents, the method comprises:
a. providing an intravaginal delivery device having a plurality of hydrogel layers, wherein adjacent hydrogel layer layers are attached to one another, wherein the layers of the plurality of hydrogel layers are different from one another by content and wherein each of at least two hydrogel layers of the plurality of hydrogel layers includes an active agent;
b. inserting the intravaginal delivery device to the vagina;
c. operating the device for a predetermined time period, thereby delivering intravaginally the plurality of active agents; and
d. withdrawing the device
wherein said operating the device comprises at least one of maintaining the device within the vagina while repeatedly inserting the device back into the vagina each time it slides out; and repeatedly withdrawing the device from the vagina and inserting it back into the vagina.

In some embodiments, the predetermined time period is within the range of 1 to 10 minutes.

In some embodiments, said delivering intravaginally the plurality of active agents is for treating a disease or disorder.

In some embodiments, there is provided use of an intravaginal delivery device having a plurality of hydrogel layers, wherein adjacent hydrogel layer layers are attached to one another, wherein the layers of the plurality of hydrogel layers are different from one another by content and wherein each of at least two hydrogel layers of the plurality of hydrogel layers includes an active agent, for the treatment of a disease or disorder.

In some embodiments, there is provided an intravaginal delivery device having a plurality of hydrogel layers, wherein adjacent hydrogel layer layers are attached to one another, wherein the layers of the plurality of hydrogel layers are different from one another by content and wherein each of at least two hydrogel layers of the plurality of hydrogel layers includes an active agent, for the treatment of a disease or disorder.

In some embodiments, there is provided a process for preparing a multi-layered intravaginal device, the process comprising the steps of:
(a) mixing water and agar, thereby obtaining an agar/water mixture;
(b) adding to the agar/water mixture an aqueous hydrogel composition comprising a plurality of naturally occurring polysaccharide and at least one cross-linking agent, thereby obtaining a hydrogel mixture;
(c) heating the hydrogel mixture to a temperature within the range of 80° C. to 100° C. while mixing;
(d) pouring the hydrogel mixture into a mold;
(e) curing the hydrogel mixture to obtain a hydrogel layer corresponding to the shape of said mold; and
(f) adhering the hydrogel layer to an additional hydrogel layer, thereby obtaining a multilayer-layered intravaginal device having two hydrogel layers.

In some embodiments, the agar concentration in the agar water mixture is within the range of 0.1 to 3% w/w.

In some embodiments, the aqueous hydrogel composition added in step (b) further includes at least one additional component selected from the group consisting of: at least one preservative, at least one stabilizer, at least one co-solvent, at least one pH modifier and at least one pharmaceutically active ingredient.

In some embodiments, heating the hydrogel mixture to a temperature within the range of 80° C. to 100° C. while mixing further includes maintaining the hydrogel mixture in said temperature for at least 15 minutes while mixing.

In some embodiments, said maintaining the hydrogel mixture in said temperature is carried out for up to 3 hours, while mixing.

In some embodiments, said adhering the hydrogel layer to an additional hydrogel layer comprises preparing a liquid form of the additional hydrogel layer and pouring said additional hydrogel layer onto the hydrogel layer.

In some embodiments, said adhering the hydrogel layer to an additional hydrogel layer comprises preparing a liquid form of the additional hydrogel layer; pouring said additional hydrogel layer into the hydrogel layer; and curing said additional hydrogel layer, wherein said hydrogel layer has the shape of a hollow elongated intravaginal device having a cavity, such that the additional hydrogel layer is poured into the cavity.

In some embodiments, said adhering the hydrogel layer to an additional hydrogel layer comprises coating the hydrogel layer with the additional hydrogel layer.

In some embodiments, said adhering the hydrogel layer to an additional hydrogel layer comprises preparing the additional layer according to steps (a) to (c); immersing the hydrogel layer obtained within the additional hydrogel layer; and curing the additional hydrogel layer.

In some embodiments, the process further comprises the step of mixing water and agar, thereby obtaining an agar/water adhesive layer; spreading the agar/water adhesive layer on the hydrogel layer prior to step (f); spreading the agar/water adhesive mixture on a surface of said hydrogel layer; and adhering the additional layer by contacting a surface thereof with the surface of said hydrogel layer.

In some embodiments, said curing is carried out for 8 to 48 hours.

Other objects, features and advantages of the present invention will become clear from the following description, examples and drawings.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIGS. 1A and 1B are schematic illustrations representing cross sections along the length and width of an elongated intravaginal delivery device, respectively, having a plurality of hydrogel layers arranged concentrically, according to some embodiments.

FIG. 1C is a top view of an elongated intravaginal delivery device having three hydrogel layers arranged concentrically, according to some embodiments.

FIGS. 2A and 2B are schematic illustrations representing cross sections along the length and width of an elongated intravaginal delivery device, respectively, having a plurality of hydrogel layers arranged in a stack, according to some embodiments.

FIG. 2C presents an elongated intravaginal delivery device having three hydrogel layers arranged in a stack, according to some embodiments.

FIG. 2D a schematic presentation of a cross sections along the width of a layer formed of a plurality of concentric layers.

FIG. 2E is a schematic presentation of a cross sections along the length of an elongated intravaginal delivery device having a plurality of hydrogel layers arranged in a stack and wrapped (or otherwise coated) with two concentric layers, according to some embodiments.

FIGS. 3A and 3B represent cross sections along the length and width of an elongated intravaginal delivery device, respectively, having a plurality of hydrogel layers embedded, and dispersed, in a leading hydrogel layer, according to some embodiments.

FIG. 4 is a scheme presenting an exemplary process for preparing a hydrogel layer forming the elongated intravaginal delivery device.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout. In the figures, same reference numerals refer to same parts throughout.

In the description and claims of the application, the words “include” and “have”, and forms thereof, are not limited to members in a list with which the words may be associated.

There is provided, according to some embodiments, an intravaginal delivery device having a plurality of hydrogel layers, wherein adjacent hydrogel layer layers are attached to one another, and wherein the layers of the plurality of hydrogel layers are different from one another by content.

The delivery capabilities of the intravaginal delivery device disclosed herein, also termed “smart vaginal hydrogel delivery system” are affected by the performative mechanical properties thereof, including, but not limited to, compressive strength, deformability modulus, and bonding strength, among others as further detailed below. The properties of the multilayered structure, including, but not limited to, density, crosslinking, concentrations of active agents, color, mechanical properties (e.g. tensile, elasticity, shore), ionic strength (pH), porosity, contents (emulsion, hydrophobic, hydrophilic) and water contents, associated by various vaginal triggers, enables to control the delivery (delivery speed, quantity delivered) of active agents, by vaginal triggers, as further detailed herein. Vaginal triggers include, but are not limited to, concentration gradients, active kinetics, pH, temperature, release time, osmosis, cross geometry and mechanical pressure.

In some embodiments, the intravaginal delivery device is having the form of a cylindrically elongated shaped stem having a distal end which is configured to be located close to the vagina opening, when the device is in use, and a proximal end configured to be directed toward the end of the vaginal canal when the device is inserted into the vagina. Typically, the distal end is wider than the proximal end. This multilayered structure, also named ‘gel stick’ and ‘smart hydrogel’ enables to deliver active agents along the vagina.

The vagina, or vagina canal, namely, the tube between the vulva and the cervix, has internal tissue wall covered by many folds, also called rugae. The tissue wall and folds of the vagina form a barrier (and access route) between the cervix and the outside world. This internal tissue of the vagina is composed of different layers of tissue: surface layers made of mucosal tissue, underneath the mucosal tissue are layers of smooth muscle tissue, collagen, and elastin fibers, which give the vagina its structure and ability to stretch. Fluids are released through the walls of the vagina to keep the area moist and, during times of sexual arousal, to increase lubrication. The vagina is also capable of absorbing some substances, such as medications, hormones (e.g. hormonal creams) or contraceptives.

Hydrogels are 3-D cross-linked hydrophilic polymer networks that can trap fluids through ‘weak interactions’, while maintaining their 3-D structure, thus having an enormous engineering potential. Hydrogels are widely used in biomedical applications. Hydrogels are highly absorbent and may contain over 90% water. However, due to the inherent cross-links, the structural integrity of a hydrogel does not dissolve or disintegrate in the presence of high concentration of water..

The term “content” with respect to hydrogels refer to the various features and components characterizing and defining a hydrogel, wherein hydrogels layers forming the device disclosed herein may differ from one another by at least one feature or component. Features and components defining hydrogels, include, for example, pH, osmolality, permeability, concentration of cross-linking agent, active ingredient(s), percentage of water, percentage of non-aqueous fluids and preservative(s), among others. The content of each hydrogel layer may lead to distinct mechanical and/or physical properties (e.g. texture, release rate of active ingredient(s) and stiffness).

The terms “active ingredient” and “pharmaceutically active ingredient” as used herein are interchangeable, and refer to a pharmaceutically active agent, composition comprising same, or a formulation comprising same that induce therapeutic activity, preferably, when administered topically, more preferably, when administered intravaginally by using the intravaginal device disclosed herein.

In some embodiments, the intravaginal device is having at least three hydrogel layers. In some embodiments, the intravaginal device is having at least four hydrogel layers. In some embodiments, the intravaginal device is having at least five hydrogel layers.

In some embodiments, the plurality of hydrogel layers includes at least one hydrophobic hydrogel layer and at least one hydrophilic hydrogel layer.

In some embodiments, the plurality of hydrogel layers includes at least two layers with different osmolality. The term “osmolality” as used herein, refers to the concentration of all solutes in a given weight of hydrogel. Accordingly, osmolality is usually expressed as units of either milliosmoles of solute per kilogram (mOsm/kg) or milliosmoles of solute per liter (mOsm/L), which is also known as osmolarity. Total solute concentration can be estimated by adding the concentrations of all individual ions and other solutes.

In some embodiments, the plurality of hydrogel layers includes at least one hydrogel layer having a first osmolality and at least one hydrogel layer having a second osmolality, wherein the first osmolality is higher than the second osmolality. It should be noted that osmolality of personal lubricants has been the focus of recent advice from the World Health Organization (WHO), in collaboration with the United Nations Population Fund and Family Health International. The WHO recommends that the osmolality of a personal lubricant should not exceed 380 mOsm/kg, in order to minimize any risk of epithelial damage; however, because most of the commercially available preparations greatly exceed this value, an upper limit of 1200 mOsm/kg is generally deemed acceptable in practice.

In some embodiments, the plurality of hydrogel layers includes at least two layers with different pH. In some embodiments, the plurality of hydrogel layers includes at least one hydrogel layer having a first pH and at least one hydrogel layer having a second pH, wherein the first pH is higher than the second pH. In some embodiments, the first pH and the second pH are acidic. In some embodiments, the first pH and the second pH are within the range of 3.8 and 4.5.

In some embodiments, the plurality of hydrogel layers includes a first hydrogel layer and a second hydrogel layer, wherein the first hydrogel layer is having a pH indicator; and the second hydrogel layer including an active agent for treating vaginal disorders related to high pH. Maintaining the pH balance of the vagina is essential for women’s health. A normal vaginal pH is usually less than 4.5 while higher pH may cause infections as it allow bacteria and yeast to thrive. Thus, one approach of determining the source of vaginal disorder, is to evaluate the vaginal pH, and if required, to modify vaginal pH, namely, reduce the pH to 4.8 or less, preferably, between 3.8 to 4.5.

A pH indicator is a compound that, when added to a solution, changes the color of the solution to a color depending on the solution pH. Commonly, the pH measured by a pH indicator is imprecise, and hence, a combination of pH indicators may be applied for achieving better accuracy. Each pH indicator operates (changes color, depending on pH) at a specific pH range. For example, methyl orange changes color from red to yellow when the pH changes from 3.2 to 4.2, respectively. In another example, azolitmin (litmus) changes color from red to blue when the pH changes from 4.5 to 8.3, respectively, hence, azolitmin incorporated in the first hydrogel layer may be blue at the beginning of the treatment with the multi-layered hydrogel device disclosed herein, and may change to red once treatment (therapeutic agent) delivered by the second layer has been completed.

Accordingly, an intravaginal delivery device that includes the first and second hydrogel layers, enables to combine pH monitoring and treatment, and further allows the user to decide, based on the color of the first hydrogel layer, whether or not to continue the treatment. Moreover, an intravaginal delivery device that includes the aforementioned first and second hydrogel layers may be subscribed to, or used by, women diagnosed with vaginal disorder related to high pH. In fact, the intravaginal delivery device can be used, or subscribed to, women diagnosed with a specific pH related disease or disorder, such that, the second layer includes a specific therapy directed to for the treatment and cure of the specific pH related disorder.

Causes of changes in vaginal pH include, but are not limited to, bacterial vaginosis, douching, menopause, Trichomonas vaginalis, group B Streptococcus (GBS), presence of menstrual blood, presence of semen, consumption of antibiotics and urinary tract infection (UTI).

In some embodiments, the second layer includes probiotics. In some embodiments, the second layer includes antimicrobial agent.

In some embodiments, the first hydrogel layer includes a plurality of first hydrogel layers. In some embodiments, the second hydrogel layer includes a plurality of hydrogel layers.

The term antimicrobial as used herein includes and/or is interchangeable with any one of the terms “antiviral”, “antibacterial” and “antifungal”.

In some embodiments, the active agent comprises a plurality of different antimicrobial agents. In some embodiments, each of the plurality of active agents is configured to exert a different antimicrobial activity relative to other active agents in the plurality of active agents. For example, the plurality of active agent may include at least one antiviral agent, at least one antifungal agent and at least one antifungal agent.

In some embodiments, the first hydrogel layer includes a pH modifying agent. Thus, in some embodiments, the pH modifying agent is an acid selected from the group consisting of citric acid, lactic acid, acetic acid, boric acid, salicylic acid, ascorbic acid, adipic acid, alginic acid and oleic acid. Each possibility represents a separate embodiment of the invention.

In some embodiments, the plurality of hydrogel layers includes at least two hydrogel layers comprising a first active agent and a second active agent, respectively. In some embodiments, the first and second active agents are different from one another. In some embodiments, the first and second active agent are the same active agent with different concentrations. For example, the first active agent is an active agent with a first concentration and the second active agent is the same active agent with a second concentration, wherein the first concentration is higher than the second concentration.

In some embodiments, the intravaginal delivery device comprises a plurality of hydrogel layers each hydrogel layer comprises an active agent, wherein each hydrogel layer is different from all other layers by the concentration of the active agent. In some embodiments, the hydrogel layers are arranged to form a decreasing or increasing concentration gradient of the active agent, across the plurality of hydrogel layers.

The terms “active agent” and “pharmaceutically active agent” as used herein are interchangeable and refer to an agent having a nutraceutical, pharmaceutical (including, diagnostic, such as, an agent for pH measurements) or therapeutic activity (including, preventive therapy and contraceptives activity), when delivered through the inner walls of the vagina from the smart hydrogel device disclosed herein. The activity of the active agent may be exerted topically, e.g. sooth a pain or an uncomfortable sensation within the vagina, and dilate a pelvic anatomical canal, and may be systemic, e.g. operate as contraceptive. It is to be understood, that the active agent may be a single agent, a combination of agents or a composition, such as, a pharmaceutical composition, comprising the active agent and non-active excipient(s), an optionally, a solvent.

In some embodiments, the active agent includes, but is not limited to, one more of the following vaginal care and/or healing agents: hormones, essential oils, chemotherapy, vitamins, local anesthetics, analgesic agents, antibacterial agents, antifungal agents, anti-itch agents, hyaluronic acid, collagen, cannabinoids, probiotics, plant (botanical) extracts, pain killers, antibiotics, dilators, lubricants, enzymes, nucleic acids, nucleotides, peptides, and vectors containing active gene/DNA fragments. Each possibility is a separate embodiment of the present invention.

In some embodiments, the cannabinoids include, but are not limited to, cannabis oils, cannabidiol (CBD), tetrahydrocannabinol (THC), such as trans-A9-tetrahydrocannabinol (THC-9) and trans-A8-tetrahydrocannabinol (THC-8), cannabidiolic acid (CBDA), tetrahydrocannabinolic acid (THCA-A), cannabigerol (CBG), cannabinol (CBN), and a combination thereof. Each possibility is a separate embodiment of the present invention.

In some embodiments, the plant extract include, but are not limited to, chamomile extract, hop extract, aloe vera extract, hemp extract, and jojoba oil. Each possibility is a separate embodiment of the present invention.

In some embodiments, the active agent includes probiotics. Probiotics refer to a supplement which comprises viable bacteria and which is capable of improving or restoring the vaginal flora. Any probiotics known in the art and suitable for vaginal application can be used. Probiotics typically include, but are not limited to, lactic acid producing bacteria, in particular genera of lactic acid bacteria, which have lactic acid as their main end product, such as selected strains of Lactobacilli, including L. acidophilus, L. jensenii, L. gasseri, L. iners, L. delbrueckii, L. plantarum, L. crispatus, L. casei, L. fermenturn, L. reuterii, L. brevis, L. salivarius, L. johnsonii L. rhamnosus; and selected strains of Bifidobacteria, including B. bifidum, B. brevi, B. adolescentis and B. longum, including mixtures of the mentioned strains. Other lactic acid producing bacteria which have been described as probiotics for vaginal application include species form Bacillus, such as B. subtilis and B. coagulans. Each possibility is a separate embodiment of the present invention.

In some embodiments, the active agent includes hormone. In some embodiments, the hormone includes, but is not limited to, estradiol, progesterone and diethylstilbestrol. Each possibility is a separate embodiment of the present invention.

In some embodiments, the active agent includes a vitamin. In some embodiments, the vitamin includes, but is not limited to, vitamin C, vitamin D, vitamin E, vitamin A, vitamin K, niacinamide (vitamin B3) and water insoluble precursors and derivatives thereof. Each possibility is a separate embodiment of the present invention.

In some embodiments, the active agent is a hydrophilic active agent. In some embodiments, the hydrophilic active agent is included within a hydrophilic hydrogel layer.

In some embodiments, the active agent is a hydrophobic active agent. In some embodiments, the hydrophilic active agent is included within a hydrophobic hydrogel layer.

In some embodiments, the active agent is an emulsion. In some embodiments, the active agent is an emulsion and the hydrogel layer including the emulsion further comprises at least one surfactant.

In some embodiments, the plurality of hydrogel layers includes at least two layers with different stiffness. The stiffness of each hydrogel layer may be controlled by the ratio between the polysaccharide and the cross-linking agent. However, other factors that affect gel stiffness, including polymerization temperature and storage duration. Gel elastic modulus, at a certain range of cross-linking agent concentration corresponds to gel stiffness. Thus, gel stiffness may be evaluated by various techniques, including through elastic modulus evaluation using, for example, Atomic Force Microscopy and force-distance curves. Thus, in some embodiments, the plurality of hydrogel layers includes at least two layers with different polysaccharide : the cross-linking agent ratios.

It is to be understood that a cross-linking agent, such as, KCl, may also act as a stabilizer, a buffer and an ion strengthening agent due to its ability to form ionic bonds. Accordingly, the terms “cross-linking agent”, “stabilizer”, “buffer” and “ion strengthening agent” as used herein are interchangeable.

In some embodiments, the plurality of hydrogel layers includes at least two layers with different cross-link density. In general, as the monomer (polysaccharide) concentration is increased, the effective density of cross-links also increases.

In some embodiments, the plurality of hydrogel layers includes at least two layers with different elasticity. Hydrogel elasticity, also known as modulus of elasticity or elastic modulus, is affected by several factors, including, cross-link density, charge density and cross-linked polymer concentration after gel preparation. Typically, hydrogels exhibit elastic moduli in the range of 0.01 to 10 kPa.

In some embodiments, the plurality of hydrogel layers includes at least two layers with different tensile characteristics. In some embodiments, the plurality of hydrogel layers includes at least two layers with different porosity.

It is to be understood that hydrogel in general are not homogenous, namely, the polymeric network from which hydrogel are formed, is not evenly distributed throughout the 3-D structure. Accordingly, the mechanical properties that differentiate between layers in the intravaginal delivery device, such as, stiffness, density and elasticity, are usually qualitative. However, the mechanical properties of the hydrogel are derived from the presence, and relative amounts, of the components forming the hydrogel, primarily, the type of polysaccharide (monomer), water, cross-linking agent, and, optionally, surfactant (required especially when oil is present), preservative (optionally) and active agent, among other optional components. The gelling conditions and process also affect the mechanical properties of the hydrogel. Thus, the difference between layers in the multi-layered device disclosed herein may be expressed quantitatively (by the presence of components and the difference in the amount(s) of one or more components) as well as qualitatively, for example, by referring to elasticity or any other mechanical property.

In some embodiments, the adjacent hydrogel layer layers are attached to one another during preparation of the intravaginal delivery device. In some embodiments, the adjacent hydrogel layer layers are attached to one another through sequential cooling. The term ‘sequential cooling’ refers to a process where the plurality of layers are not cooled at the same time, but rather, one layer is cooled and solidifies, thereafter another layer is cooled and solidifies, and so on. Alternatively, the adjacent hydrogel layer layers are attached to one another by pressing adjacent layer one to another at the pre-gelled state, and continuing the press until the layers are cured/solidify.

The term ‘cured’ as used herein with respect to a hydrogel layer refer to a hydrogel layer that is hard/solid/soft solid. Curing is typically induced by heat or radiation, and may be accompanied by compression under high pressure, or by mixing with a curing catalyst. Curing is irreversible hardening of thermoset polymers. The term ‘cure’ as used herein with respect to a hydrogel layer is exchangeable with the terms ‘set’ and ‘molded’.

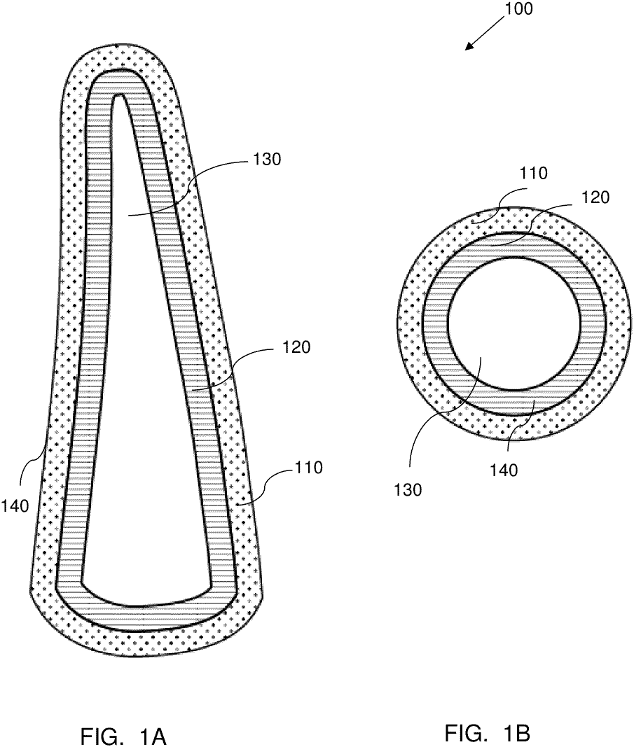

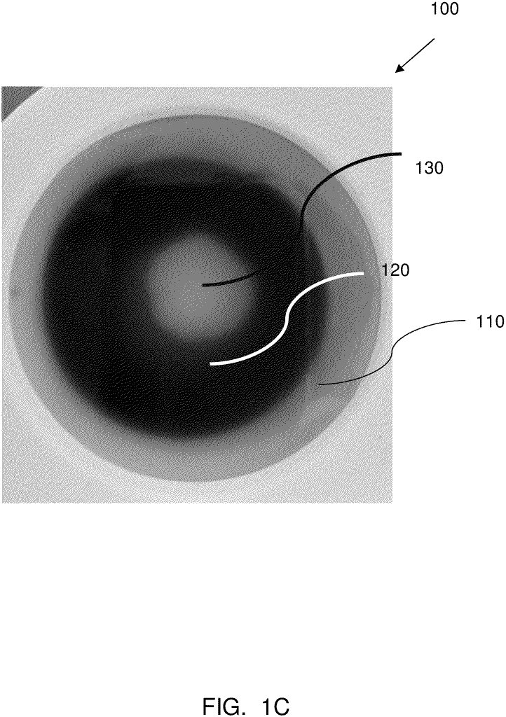

Reference is now made to FIGS. 1A and 1B, which constitute perspective views of an intravaginal delivery device 100, according to some embodiments and to FIG. 1C which exhibits a top view of an elongated intravaginal delivery device having three hydrogel layers arranged concentrically. Intravaginal delivery device 100 includes three hydrogel layers arranged in a concentric structure, as follows: external hydrogel layer 110, transitional hydrogel layer 120 and core hydrogel layer 130, wherein the layers are different from one another by content. Intravaginal delivery device 100 further has a device external surface 140, which is a hydrogel layer configured to be in direct contact with the vagina, upon inserting intravaginal delivery device 100 to the vagina. In some embodiments, device external surface 140 is hydrogel layer 110. Stated otherwise, device external surface 140 is an integral part of hydrogel layer 110, and not a separate layer. The internal surface of external hydrogel layer 110 is in direct contact with the external surface of transitional layer 120. In some embodiments, transitional layer 120 includes a first active agent, the transition of which from transitional layer 120 through external hydrogel layer 110 to the vagina is diffusion mediated. In some embodiments, the transition of the first active agent from transitional layer 120 to the vagina is afforded by osmosis.

In some embodiments, device external surfaces 140 is hydrogel layer 110, which includes at least one dilator. Device external surfaces 140 is configured to ease the insertion of the intravaginal delivery device into the vagina. In some embodiments, device external surfaces 140 is a thin layer that disintegrates upon entering the vagina, or soon after, thereby exposing the more internal hydrogel layer (e.g layer 120) to the vagina, and enabling direct contact of hydrogel layer 120 within the vagina. This set up enables transition of active ingredients from the internal layers directly to the vagina without the need to include therein lubricants/dilators/local anesthetics. In some embodiments, device external surfaces 140 includes a therapeutically active ingredient or a composition comprising same.

In some embodiments, the first active agent is stained, such that, the transition of which along the layers is visible. For example, the transition layer has a first color rendered by the presence of first active agent, therewithin and the external layer is colorless. Thus, upon transition of the first active agent across the external layer, the color of the transition layer fades, indicating that reduction in amount of the first active agent, while the external layers begin to obtain the first color, as the first active agent is entering this layer. Similarly, any active agent crossing the layers, e.g. from the core layer to the vagina through the transition and external layers, may be stained with a first color, such that as it travels across the layers, the first color in the layer where the active ingredient is initially present, begins to fade while the layers where the active agent travels through acquire the first color during the presence of the active ingredient therein. This staining mechanism may be applied for monitoring the diffusion during storage, or following use or multiple uses, where the disappearance of the stain from all layers, indicates that the device should not be further used, as the active agent has been exhausted.

In some embodiments, at least one layer of the plurality of layers is a hybrid hydrogel layer, including a hydrogel layer having non-hydrogel particles embedded therein. In some embodiments, the non- hydrogel particles are nanoparticles. In some embodiments, the non-hydrogel particles are microparticles. In some embodiments, the non-hydrogel particles are core-shell particles. In some embodiments, the non-hydrogel particles are liposomes encapsulating at least one active ingredient. In some embodiments, the at least one encapsulated active agent is a drug. In some embodiments, the at least one encapsulated active agent is a peptide. In some embodiments, the non-hydrogel particles are poly(vinyl alcohol) (PVA) microcapsules encapsulating chamomile extract.

In some embodiments, core hydrogel layer 130 includes a second active agent configured to move across core layer 130, transitional layer 120 and external layer 110 to the vagina. In some embodiments, the transition of the second active agent across core layer 130, transitional layer 120 and external layer 110 to the vagina is diffusion mediated. In some embodiments, the transition of the second active agent is afforded by osmosis. In some embodiments, the transition of the second active agent across core layer 130, transitional layer 120 and external layer 110 to the vagina is triggered by mechanical pressure induced by the vaginal walls and/or by the force applied when squeezing intravaginal delivery device 100 into the vagina.

Although FIG. 1A depicts transitional layer 120 and external layer 110 as having the same width/thickness, each of these layers may have a different thickness. Furthermore, although FIGS. 1A and 1B depict intravaginal delivery device 100 as having three (3) layers, intravaginal delivery device 100 may have two layers, or four layers, or at least five layers.

In some embodiments, transition of any one of the first and second active agents across the layers of intravaginal delivery device 100 is induced by the pressure created during insertion of intravaginal delivery device 100 into the vagina. In some embodiments, transition of any one of the first and second active agents across the layers of intravaginal delivery device 100 is induced by vaginal triggers, as body temperature, namely, the temperature at the vagina. In some embodiments, transition of any one of the first and second active agents across the layers of intravaginal delivery device 100 is induced by a temperature within the range of 35 to 39 degree C. In some embodiments, transition of any one of the first and second active agents across the layers of intravaginal delivery device 100 is induced by contact with the vagina. Contact of the outer layer of external layer 110 with the vagina maybe associated with some friction and/or pressure created between the vagina wall and the device, which may induce transition of any one of the first and second active agents across the layers of intravaginal delivery device 100. In some embodiments, transition of any one of the first and second active agents across the layers of intravaginal delivery device 100 does not occur, or occurs slowly and insignificantly, during storage of the device, when not in use. Lack or reduced transition across layers during storage, may be afforded due to lack of mechanical pressure on the device, the pressure which is induced when holding/squeezing the device and also when the device is pressed within the walls of the vaginal canal. Alternatively, or in addition, lack or reduced transition across layers during storage, may be achieved by maintaining the device at low temperatures during storage. Low temperatures may be any temperature below 32 degree C., for example, room temperature (about 25° C.) or lower.

In some embodiments, the plurality of layers of intravaginal delivery device 100 are configured to provide slow release of active ingredient(s) across the layers, when the intravaginal delivery device 100 is within the vagina. In some embodiments, each hydrogel layer 110, 120 and 130 includes an active ingredient with differential release properties.

In some embodiments, external layer 110 includes an immediate release composition of the active ingredient, transitional layer 120 includes a slow-release composition of the active ingredient and core layer 130 includes a composition of the active ingredient the release rate of which is slower than the release rate of active agent from the slow release composition in transitional layer 120.

In some embodiments, external layer 110 includes an agent configured to change the vaginal pH to a required pH, wherein the required pH is the pH required for activating an active agent stored in transitional layer 120 and/or in core layer 130. In some embodiments, external layer 110 includes an agent configured to change the vaginal pH to a required pH, wherein the required pH is the pH required for inducing transition of an active agent stored in transitional layer 120 and/or in core layer 130 across said layers towards contact with the vaginal walls. In some embodiments, external layer 110 has the required pH, wherein the required pH is the pH required for activating an active agent stored in transitional layer 120 and/or in core layer 130. In some embodiments, external layer 110 has the required pH, wherein the required pH is the pH required for inducing transition of an active agent stored in transitional layer 120 and/or in core layer 130 across said layers towards contact with the vaginal walls.

It is to be understood that the smart vaginal hydrogel delivery system and the active agent composition are designed to meet vaginal triggers, in order to achieve any desired rate of delivery and amount of delivered active agent. Thus, the hydrogel layers composing the intravaginal delivery device, and the active agent composition are designed such that rates of release and of delivering to the vagina (across the hydrogel layer), of each active agent, is adapted to the time that the intravaginal delivery device stays in the vagina. Typically, the intravaginal delivery device is configured to stay in the vagina a few minutes. Thus, for active agent that require immediate delivery in one use (application), the plurality of layers are designed to deliver the active ingredient(s) enclosed therein, within a few minutes. In the event that the intravaginal delivery device is configured for multiple uses, where in each use specific amount and/or active agent has to be released and delivered to the vagina, the hydrogel layers composing the device may be designed such that the outer layer (e.g. external layer 110) delivers on the first use some or all of the active ingredient(s) enclosed therein, but the remaining, more internal, layers do not deliver the active ingredient on the first use, but only on consecutive uses. This may be afforded by including in the internal layers slow-release compositions designed for slow delivery in accordance with treatment regimen.

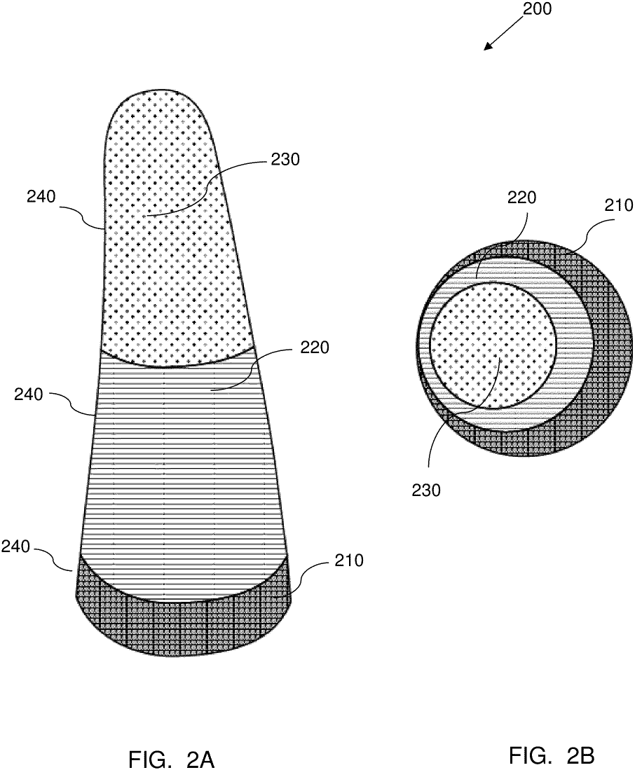

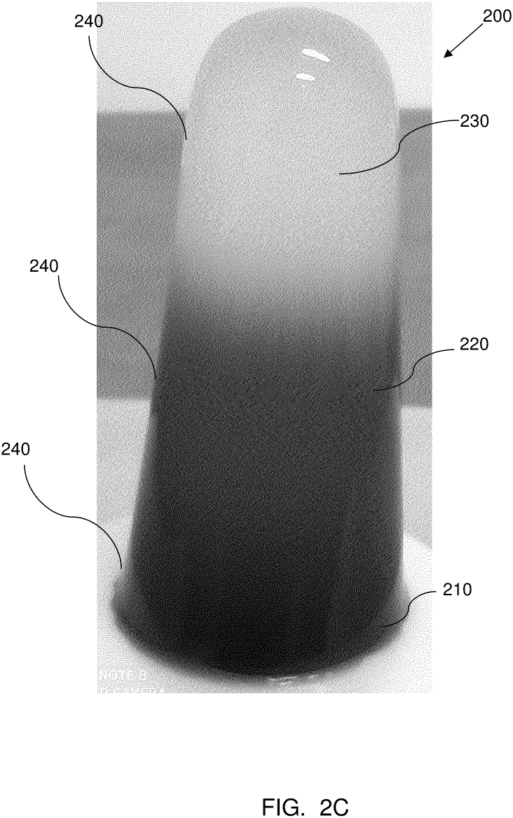

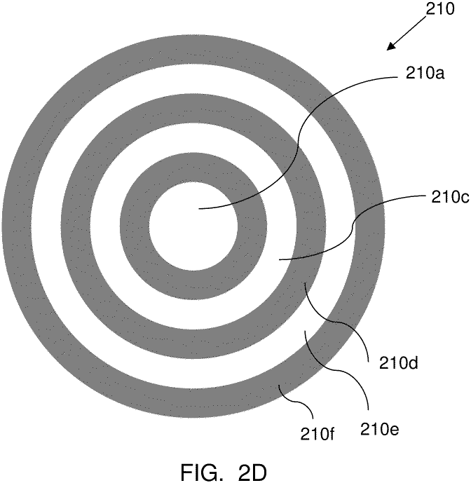

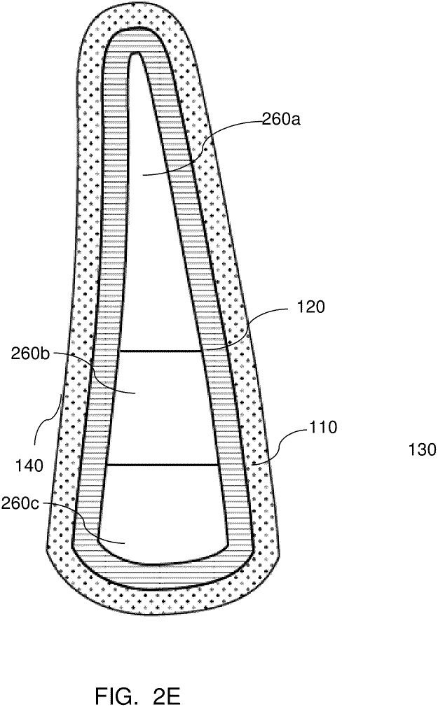

Reference is now made to FIGS. 2A to 2E, which constitute perspective views of intravaginal delivery device 200, according to some embodiments. In some embodiments, intravaginal delivery device 200 includes three hydrogel layers arranged in a stack structure, as follows: a bottom layer 210, a middle layer 220 and a top layer 230, wherein the layers are different from one another by content. Intravaginal delivery device 200 may further have a device external surface 240 which is a hydrogel layer configured to be in direct contact with the vagina, upon inserting intravaginal delivery device 200 to the vagina. In some embodiments, device external surface 240 corresponds to the external surface of the layers composing intravaginal delivery device 200, namely, bottom layer 210, middle layer 220 and top layer 230. In some embodiments, device external surface 240 is a separate layer enveloping intravaginal delivery device 200, as shown for example in FIG. 2E. In some embodiments, device external surface 240 is a plurality of separate layers enveloping intravaginal delivery device 200, as shown for example in FIG. 2E. Bottom layer 210 is at the distal end of intravaginal delivery device 100 and is having a bottom surface facing the environment and a top surface in contact with the bottom of middle layer 220. Typically, the structure of the device is conical, and hence, the perimeter of the bottom surface of the bottom layer is larger than all other perimeters of the device. The bottom surface of middle layer 220 is in contact with the top surface of bottom layer 210 and the top surface of middle layer 220 is in contact with the bottom surface of top layer 230. Top layer 230 is at the proximal end of intravaginal delivery device 100 and is typically the narrower layer thereof.

In some embodiments, at least one layer of the stacked layers is a concentric layer formed of a plurality of concentric layers. For example, as shown in FIG. 2D, bottom layer 210 comprises six concentric layers 210a-210f, wherein each concentric layer of the bottom layer is distinct. In another example, the intravaginal delivery device includes three hydrogel layers arranged in a concentric structure, as shown in FIG. 1A with the exception that the core hydrogel layer (termed core layer 130 in FIG. 1A) includes three hydrogel layers 260a-260c, arranged one on top of the other, in a stack (similar to the structure of delivery device 200), as shown in FIG. 2E. Thus, the smart device disclosed herein may include a combination of structures, such as, a combination of concentric and stack structures, to address the various vaginal triggers, and hence provide additional options for delivering a large variety of active agents, at various timings of release and/or at various compositions that can be more easily delivered from separate layers, as their combination may reduce their efficiency and/or their activity.

In some embodiments, intravaginal delivery device 200 includes an active agent and/or a pharmaceutical composition comprising same in top layer 230, wherein the active agent is configured to ease the penetration of intravaginal delivery device 200, wherein the remaining layers include active ingredient adapted for vaginal care of therapy. In some embodiments, the active agent in top layer 230 is selected from an anesthetic and pain relief agent, and the active agent in at least one of the remaining hydrogel layers is, for example, a blood-clot promoting drug, such as, tranexamic acid.

In some embodiments, intravaginal delivery device 200 comprises an additional external hydrogel surface layer covering device external surface 240. In some embodiments, the additional external hydrogel surface layer does not cover the bottom surface of bottom layer 210. Thus, in some embodiments, intravaginal delivery device 200 is composed of stacked layers and is wrapped by at least one additional layer, similar in structure to external layer 110 of intravaginal delivery device 100. Thus, the smart device disclosed herein may combine a multi-layered stack structure with additional one or more layers enveloping the stacked structure. This combined structure can be useful when requiring an immediate contact with the vagina over the entire peripheral length and width of the device, e.g. when the outer layer comprises a lubricant, local anesthetic and/or a dilator, which ease the insertion of the device into the vagina, while the other stacked layer can exert their activity once the device is inside the vagina. In some embodiments, the additional external surface layer is a thin layer that disintegrates upon entering the vagina, or soon after, thereby exposing the stacked layers to the vagina, and enabling direct contact thereof with the vagina. This set up also enables transition of active ingredients from the stacked layer directly to the vagina without the need to diffuse through the cover layer.

The term ‘wrapped’, ‘coated’ and ‘covered’ as used herein, are interchangeable, and refer to a layer that envelopes, otherwise wraps/coats/covers another layer. Typically, the coating layer is relatively thin. In some embodiments, the coating layer is having a thickness within the range of 10 µm to 10 mm, 50 µm to 10 mm, 100 µm to 10 mm, 10 µm to 8 mm, 10 µm to 5 mm, 10 µm to 3 mm, 10 µm to 1 mm, 50 µm to 8 mm, 50 µm to 5 mm, 50 µm to 3 mm, 50 µm to 1 mm, 100 µm to 2 mm, 100 µm to 3 mm or 100 µm to 5 mm.

In some embodiments, intravaginal delivery device comprises a plurality of layers arranged in a stack structure and further comprises at least one cover layer, which covers the external surface of intravaginal delivery device. Alternatively, or in addition, at least one of the stacked layers comprises a plurality of concentric layers.

In some embodiments, at least one layer of the plurality of stacked layers is a hybrid hydrogel layer, including a hydrogel layer having non-hydrogel particles embedded therein. In some embodiments, the non-hydrogel particles are nanoparticles. In some embodiments, the non-hydrogel particles are microparticles. In some embodiments, the non-hydrogel particles are liposomes comprising an active ingredient. In some embodiments, the active agent is a drug. In some embodiments, the active agent is a peptide.

It is to be understood that although FIGS. 2A - 2C and 2E represent intravaginal delivery device including three stacked layers, any other number of layers, such as, two layers, four layers or at least five layers, stacked one on top of the other, may be included in the intravaginal delivery device. Furthermore, while FIGS. 2A and 2C exhibit bottom layer 210 thinner (shorter) than each of middle layer 220 and top layer 230, intravaginal delivery device may have any other configuration, including a configuration where all stacked layers have the same height (thickness) and the like.

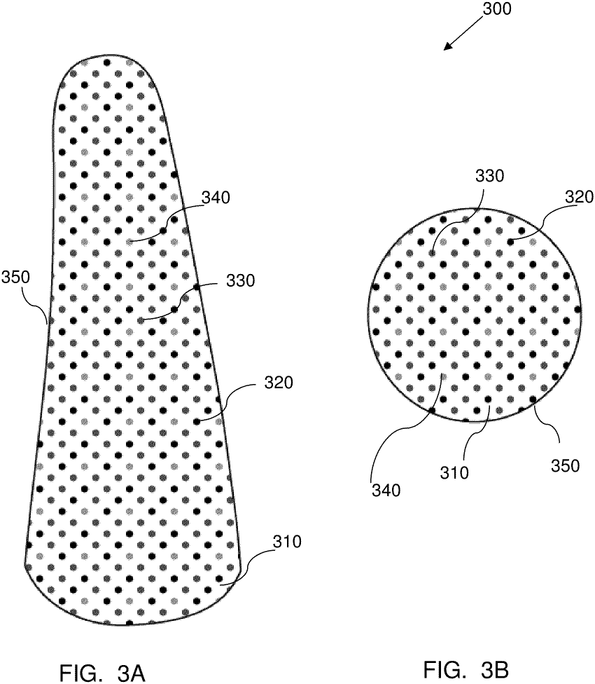

Reference is now made to FIGS. 3A to 3B, which constitute perspective views of intravaginal delivery device 300, according to some embodiments. Intravaginal delivery device 300 includes a foundational layer 310 which provides intravaginal delivery device 300 its 3-D elongated concentric structure. Intravaginal delivery device 300 further includes a plurality of layers 320, 330 and 340, dispersed within foundational layer 310. In some embodiments, the plurality of layers 320, 330 and 340 are in the form of particles. In some embodiments, the plurality of layers dispersed with the foundational layer are randomly dispersed therein. In some embodiments, the plurality of layers dispersed with the foundational layer are dispersed orderly. For example, particle forming layer 320 may be located at the perimeter of intravaginal delivery device 300 while the other particles may be located at the center of intravaginal delivery device 300. In some embodiments, the particles of at least one layer are nanoparticles. In some embodiments, the particles of at least one layer are microstructures. In some embodiments, each of the plurality of layers dispersed with the foundational layer encapsulate an active agent. In some embodiments, intravaginal delivery device 300 further includes device external layer 350, which is a hydrogel layer configured to be in direct contact with the vagina, upon inserting intravaginal delivery device 300 to the vagina. In some embodiments, device external surface 350 is hydrogel layer 310. Stated otherwise, in some embodiments, device external surface 350 is an integral part of hydrogel layer 310, and not a separate layer. In some embodiments, device external surface 350 is a separate layer enveloping intravaginal delivery device 300.

In some embodiments, device external surfaces 350 is hydrogel layer 310, which includes at least one dilator. In some embodiments, device external surfaces 350 is hydrogel layer 310, which includes at least one local anesthetic. Device external surfaces 350 is configured to ease the insertion of the intravaginal delivery device into the vagina.

In some embodiments, each hydrogel layer includes at least one naturally occurring polysaccharide, water and a cross-linking agent. In some embodiments, each hydrogel layer includes a plurality of naturally occurring polysaccharides, water and a cross-linking agent. In some embodiments, each hydrogel layer includes at least three naturally occurring polysaccharide, water and a cross-linking agent.

The term ‘plurality’ as used herein refers to values of more than one, or of at least two.

In some embodiments, each hydrogel layer includes a plurality of naturally occurring polysaccharides, water, at least one cross-linking agent and at least one pharmaceutically active agent, wherein the at least one cross-linking agent may also be used as an ion strength agent.

In some embodiments, each hydrogel layer includes a plurality of naturally occurring polysaccharides, water, at least one cross-linking agent, at least one preservative and at least one ion strength agent.

In some embodiments, each hydrogel layer includes a plurality of naturally occurring polysaccharides, water, at least one cross-linking agent, and at least one co-solvent.

In some embodiments, each hydrogel layer includes a plurality of naturally occurring polysaccharides, water, at least one cross-linking agent, and at least one pH modifying agent.

In some embodiments, each hydrogel layer includes a plurality of naturally occurring polysaccharides, water, at least one cross-linking agent, and at least one surfactant.

In some embodiments, each hydrogel layer includes a plurality of naturally occurring polysaccharides, water, at least one cross-linking agent and, optionally, one or more of the following: at least one preservative, at least one co-solvent, at least one pH modifying agent, at least one surfactant and at least one pharmaceutically active agent.

In some embodiments, the amount of the plurality of naturally occurring polysaccharides in each hydrogel layer is within the range of 1 - 5% w/w. In some embodiments, the amount of the plurality of naturally occurring polysaccharides in the elongated intravaginal device is within the range of 1 - 5% w/w.

In some embodiments, the amount of the at least one cross-linking agent in each hydrogel layer is within the range of 0.4 - 2% w/w. In some embodiments, the amount of the at least one cross-linking agent is within the range of 0.4 - 2% w/w.

In some embodiments, the amount of the at least one preservative in each hydrogel layer is within the range of 0.05 - 1% w/w. In some embodiments, the amount of the at least one preservative is within the range of 0.05 - 1% w/w.

In some embodiments, the amount of the at least one co-solvent in each hydrogel layer is within the range of 1 - 5% w/w. In some embodiments, the amount of the at least one co-solvent is within the range of 1 - 5% w/w.

In some embodiments, the amount of the at least one pH modifying agent in each hydrogel layer is within the range of 0.1 - 3% w/w. In some embodiments, the amount of the at least one pH modifying agent is within the range of 0.1 - 3% w/w.

In some embodiments, the amount of the at least one surfactant in each hydrogel layer is within the range of 2% w/w. In some embodiments, the amount of the at least one surfactant is within the range of 0.1 - 2% w/w.

In some embodiments, the naturally occurring polysaccharide includes, but is not limited to, any one of the following naturally occurring polysaccharide: collagen, locust bean gum, carrageenan, i-carrageenan (iota-carrageenan), low acyl gellan gum, high acyl gellan gum, agar, gum karaya, gum Arabic, gum tragacanth, guar gum, konjac gum, pectin, xanthan gum, welan gum, native or modified starch, inulin, a cellulose derivative, chitin, chitosan, alginate, hyaluronic acid, pectin, and a combination thereof. Each possibility is a separate embodiment of the present invention.

Gellan gum is a high-molecular-weight extracellular polysaccharide produced from the fermentation of a carbohydrate by strains of Pseudomonas elodea. Gellan gum is divided into two types, high acyl and low acyl form by the degree/percent of substitution by O-acyl groups. The high acyl Gellan gum, also called HA Gellan gum, has two acyl substituents acetate at C6 and glycerate at C2 on the first glucose unit of the repeating unit of tetrasaccharide, and on average, there is one glycerate per repeat and one acetate per every two repeats. The low acyl Gellan gum, also called LA Gellan gum, is partly deacylated or fully deacylated, where its common form is the fully deacylated one, with no detectable acyl groups, also called deacetylated gellan gum. In general, HA Gellan gum forms soft, elastic and non-brittle gels whereas LA Gellan gum forms firm, non-elastic and brittle gels.

In some embodiments, the water in each hydrogel layer is in an amount of at least 60% by weight, relative to the weight of the hydrogel layer. In some embodiments, the water in each hydrogel layer is in an amount within the range of about 60% to about 90% by weight, about 70% to about 80% by weight, or about 80% to about 90% by weight, relative to the weight of the hydrogel layer. Each possibility is a separate embodiment of the present invention.

In some embodiments, the cross-linking agent includes a pharmaceutically acceptable cross-linking agent. The pharmaceutically acceptable cross-linking agent is a salt of a monovalent cation or a multivalent cation such as a salt of a divalent cation, a trivalent cation or a tetravalent cation. The cross-linking agent may be further act as a stabilizer, a buffer and/or an io-strengthening agent.

Examples of salts of monovalent cations include, but are not limited to, sodium chloride and potassium chloride. Examples of salts of divalent cations include, but are not limited to, calcium chloride, calcium sulfate, calcium phosphate, calcium carbonate, magnesium chloride, magnesium sulfate, magnesium phosphate, magnesium carbonate, manganese chloride, manganese sulfate, manganese phosphate, manganese carbonate, zinc chloride, zinc sulfate, zinc phosphate, and zinc carbonate. Examples of trivalent or tetravalent cations include, but are not limited to, Al³⁺, Fe³⁺ Ce^{3+,} Sn⁴⁺ Zr⁴⁺, and Ti⁴⁺. In some embodiments, the cross-linking agent is selected from the group consisting of trisodium citrate, glucose, mannose and mannitol. The latter may act as stabilizers.

In some embodiments, the cross-linking agent is selected from the group consisting of salts of monovalent cations, divalent cations, trivalent cations, quadrivalent cations, and a combination thereof. According to further embodiments, the cross-linking agent is a salt of a monovalent cation, a divalent cation, or a combination thereof. According to a certain embodiment, the cross-linking agent is a mixture of sodium chloride and potassium chloride or a mixture of calcium chloride and potassium chloride. According to some embodiments, the amount of the cross-linking agent is up to about 10% by weight relative to the weight of a hydrogel layer. Alternatively, the amount of the cross-linking agent ranges from about 0.1% to about 5% by weight relative to the weight of a hydrogel layer, or from about 0.1% to about 1%, or from 0.2% to about 0.5% by weight relative to the weight of a hydrogel layer.

In some embodiments, the at least one hydrogel layer may further include at least one pharmaceutically acceptable co-solvent. In some embodiments, the at least one pharmaceutically acceptable co-solvent includes, but is not limited to, glycerol and polyethylene glycols (PEGs).

In some embodiments, the at least one hydrogel layer may further include at least one pharmaceutically acceptable surfactant. In some embodiments, the amount of surfactant in the at least one layer may be at most 5% by weight, relative to the weight of the at least one hydrogel layer.

The term “pharmaceutically acceptable” as used herein refers to compounds that are approved by a regulatory agency of the Federal or a state government or listed in the U.S. Pharmacopeia or other generally recognized pharmacopeia for use in humans. Each and every component of the intravaginal delivery device disclosed herein is a pharmaceutically acceptable component.

Surfactants, also known as surface-active agents are compounds capable of lowering surface tension between two materials, such as, oil and water. Thus, in the presence of a suitable surfactant, oil and water form an emulsion. Surfactants may be used as detergents, emulsifiers and dispersants among other uses. A hydrophilic/lipophilic balance (HLB) of a surfactant is a parameter indicating the surfactant’s affinity toward water or oil. HLB scale ranges from 1 (totally lipophilic) to 20 (totally hydrophilic), with 10 representing an equal balance of both characteristics.

Thus, in some embodiments, the at least one pharmaceutically acceptable surfactant has an HLB between about 3 to about 16. In some embodiments, the at least one pharmaceutically acceptable surfactant has an HLB between about 9 to about 14. The aforementioned HLB values refer to HLB required for stabilizing an oil-in-water (O/W) emulsion of a given oil.

As used herein, the term “about” may be used to specify a value of a quantity or parameter within a continuous range of values in the neighborhood of (and including) a given (stated) value. According to some embodiments, “about” may specify the value of a parameter to be between 80% and 120% of the given value. For example, the statement “HLB of about 10” is equivalent to the statement “HLB of about 8 to 12”. According to some embodiments, “about” may specify the value of a parameter to be between 90% and 110% of the given value. According to some embodiments, “about” may specify the value of a parameter to be between 95% and 105% of the given value.

In some embodiments, the at least one pharmaceutically acceptable surfactant is a nonionic surfactant. In some embodiments, the at least one pharmaceutically acceptable surfactant is an anionic surfactant. In some embodiments, the at least one pharmaceutically acceptable surfactant is a cationic surfactants. In some embodiments, the at least one pharmaceutically acceptable surfactant is an amphoteric surfactants. In some embodiments, the at least one pharmaceutically acceptable surfactant includes a plurality of surfactants. In some embodiments, the plurality of surfactants may be of the same type (e.g. anionic or cationic). In some embodiments, the plurality of surfactants may be of different types.

Exemplary nonionic surfactants include, but are not limited to, polysorbate, sorbitan fatty acid esters, polyoxysorbitan fatty acid esters, polyoxyalkylene higher alcohol ethers, and polyoxyalkylene higher alcohol esters. Thus, nonionic surfactants include polyoxyethylene sorbitol esters such as polyoxyethylene sorbitan monolaurate (Tween 20), polyoxyethylene sorbitan monostearate (Tween 60) and polyoxyethylene sorbitan monooleate (Tween 80); Tyloxapol; polyoxyethylene isooctylphenyl ethers such as Triton X-100, poly(oxyethylene) nonylphenyl ethers such as NP-40, poly(oxyethylene) cetyl ether, poly(oxyethylene) palmityl ether; octyl glucoside, and alkyl maltoside such as n-dodecyl-beta-D-maltoside; Poloxamer 4070; Poloxamer 188; polyoxyl 40 stearate; glyceryl stearate; polyoxyethylene (POE) fatty acid esters, such as Myrj 45, Myrj 49, Myrj 52 and Myrj 59; poly(oxyethylene) alkylyl ethers, such as poly(oxyethylene) cetyl ether, poly(oxyethylene) palmityl ether, polyethylene oxide hexadecyl ether, poly(oxyethylene) dodecyl ethers such as Brij 58; polyethylene glycol cetyl ether; polyglyceryl oleate; lecithin; and any combination thereof. Each possibility is a separate embodiment of the invention. TWEEN® and poloxamer surfactants are preferred as they are FDA approved for human use.

Further exemplary nonionic surfactants include, but are not limited to, fatty alcohol ethoxylates (alkylpolyethylene glycols); alkylphenol polyethylene glycols; alkylmercaptan polyethylene glycols; fatty amine ethoxylates (alkylaminopolyethylene glycols); fatty acid ethoxylates (acylpolyethylene glycols); polypropylene glycol ethoxylates (Pluronics™; e.g., Pluronic F-68); fatty acid alkylol amides, (fatty acid amide polyethylene glycols); N-alkyl-, N-alkoxypoly-hydroxy-fatty acid amide, sucrose esters; sorbitol esters and polyglycol ethers, polyoxyethylene-hydrogenated castor oil, fatty acid alkanolamide, sucrose fatty acid esters, glycerol mono, di- and trioctanoate. Each possibility is a separate embodiment of the invention.

Exemplary anionic surfactants include, but are not limited to, alkyl sulfates, olefin sulfates, ether sulfates, monoglyceride sulfates, alkyl sulfonates, aryl sulfonates, olefin sulfonates, alkyl sulfosuccinates, aryl sulfosuccinates, including sodium dodecyl sulphate (SDS), dioctyl sodium sulfosuccinate, dioctyl sodium sulfonate. Each possibility represents a separate embodiment of the invention.

Exemplary cationic surfactants include, but are not limited to, benzalkonium salts, polyoxyalkylene alkylamines, alkylamines, alkanolamine fatty acid esters, quaternary ammonium fatty acid esters, dialkyl ammonium salts, alkyl pyridinium salts including stearylamine, triethanolamine oleate, benzethonium chloride. Each possibility represents a separate embodiment of the invention.

Exemplary amphoteric surfactants include, for example, imidazoline-based amphoteric surfactants (such as a 2-cocoyl-2-imidazoliniumhydroxide-1-carboxyethyloxy-2-sodium salt), betaine-based surfactants (such as alkyl betaine, amide betaine, and sulfo betaine), and acylmethyl taurine. Each possibility represents a separate embodiment of the invention.

In some embodiments, at least one hydrogel layer may further include oil. In some embodiments, at least one hydrogel layer may further include up to 40% oil by weight, relative to the weight of the at least on hydrogel layer. In some embodiments, the oil is a naturally occurring oil. In some embodiments, the oil is selected from a vegetable oil, an animal oil and a mineral oil. In some embodiments, the naturally occurring oil is a vegetable oil selected from the group consisting of coconut oil, corn oil, sesame seed oil, sunflower oil, walnut oil, canola oil, castor oil, olive oil, peanut oil, safflower oil, shea oil, and a mixture thereof. Each possibility represents a separate embodiment of the invention.

In some embodiments, the naturally occurring oil is present in an amount of up to about 40% by weight of a hydrogel layer comprising same, alternatively the naturally occurring oil is present in an amount ranging from about 5% to about 30% by weight of a hydrogel layer comprising same, or any integer in between, thus forming an oil-in-water emulsion prior to gelation when a surfactant is present.

In some embodiments, at least one hydrogel layer may further include at least one pharmaceutical acceptable preservative. In some embodiments, at least one pharmaceutical acceptable preservative is selected from parabens, phenoxyethanol, salts of benzoic acid, sorbic acid or citric acid such as potassium sorbate, sodium benzoate or trisodium citrate, sodium metabisulfite, disodium EDTA, benzalkonium chlorobutanol and a combination thereof.

In some embodiments, there is provided a kit, the kit includes the intravaginal delivery device disclosed herein and a case configured to enclose the device therewithin. In some embodiments, the case is a solid cover.

In some embodiments, the case is configured to hermetically enclose the device therewithin, in order to prevent drying of the intravaginal delivery device. It is to be understood that the device may have some degree of humidity, which maintains it semi-flexible, and easy to insert into the vagina. Hence, to protect the device from losing its humidity, a case can be used. The case may be also used for maintaining the device geometry, namely, the 3-D elongated structure of the device. In some embodiments, the case is configured for maintaining the device clean after being washed between uses. The case may be also used for storing the device therein during shipment and marketing.

In some embodiments, the kit further includes instructions for use.

In some embodiments, the kit further includes a pH indicator and a corresponding manual presenting the colors of the pH indicators vs the corresponding pH values.

In some embodiments, there is provided a method for intravaginal delivery of a plurality of active agents, the method includes
(a) providing an intravaginal delivery device having a plurality of hydrogel layers, wherein adjacent hydrogel layer layers are attached to one another, wherein the layers of the plurality of hydrogel layers are different from one another by content and wherein each of at least two hydrogel layers of the plurality of hydrogel layers includes an active agent;
(b) inserting the intravaginal delivery device to the vagina;
(c) operating the device for a predetermined time period, thereby delivering intravaginally the plurality of active agents; and
(d) withdrawing the device,
wherein said operating the device includes maintaining the device within the vagina while repeatedly inserting the device back into the vagina each time it slides out, or repeatedly withdrawing the device from the vagina and inserting it back into the vagina.

In some embodiments, the intravaginal delivery device is having the form of a cylindrically elongated shaped stem having a narrow end and a wide end, wherein said inserting includes holding the wide end while inserting the narrow end to the vaginal opening and pushing the device into the vaginal canal.

In some embodiments, said maintaining the device within the vagina includes in-out motion, namely, pushing the device into the vagina (while holding the wide end of the device), and withdrawing it out, or letting the device slide out (by forces applied thereon by the vaginal walls/muscles) and then pushing it back in.

It is to be understood that inserting the device into the vagina when holding the device at its wide end, also termed herein ‘base’, the base is pressed towards the vaginal opening and the device penetrates into the vagina. While the device is within the vagina the vagina walls/muscle create resistance and enable dilation therapy by penetration into vagina. When the pressure at the base is relaxed a natural contraction from the vaginal muscles, pushes the device out. This repeat motion of applying pressure at base so the intimate gel stick simulates penetration also provides intimate training.

The predetermined time period for maintaining the device in the vagina depends on the active ingredients and the effect exerted thereby which is required to achieve. Typically, the time period should be in the range of several minutes. In some embodiments, the predetermined time period is within the range of 1 to 10 minutes. In some embodiments, the predetermined time period is within the range of 2 to 9 minutes. In some embodiments, the predetermined time period is within the range of 3 to 7 minutes.

In some embodiments, the active agent is an active agent for vaginal use. In some embodiments, the active agent is an FDA approved active agent for vaginal use. In some embodiments, the active agent is selected from the group consisting of butoconazole nitrate, candicidin clindamycin phosphate, clotrimazole, dienestrol, diethylstilbestrol, dinoprostone, estradiol, estradiol acetate, conjugated estrogens, estropipate, ethinyl estradiol, etonogestrel, gentian violet, hydrocortisone, metronidazole, miconazole nitrate, miconazole nitrate, nonoxynol-9, nystatin, oxytetracycline hydrochloride (polymyxin b sulfate), prasterone, progesterone, sulfanilamide, terconazole, tioconazole, triple sulfa (sulfabenzamide;sulfacetamide;sulfathiazole). In some embodiments, the active agent is a contraceptive. In some embodiments, the contraceptive is a spermicide.

In some embodiments, there is provided a method for treating a disease or disorder, the method includes
(a) providing an intravaginal delivery device having a plurality of hydrogel layers, wherein adjacent hydrogel layer layers are attached to one another, wherein the layers of the plurality of hydrogel layers are different from one another by content and wherein each of at least two hydrogel layers in the plurality of hydrogel layers includes active agent for treating a disease or disorder;
(b)inserting the intravaginal delivery device to the vagina;
(c) maintaining the device within the vagina for a predetermined time period thereby delivering intravaginally the active agent of each of the at least two hydrogel layers; and
(d)withdrawing the device.

In some embodiments, steps (b) to (d) are repeated at least one more time. In some embodiments, steps (b) to (d) are repeated at least once a day, for at least two days. In some embodiments, steps (b) to (d) are repeated at least once a day, for at least two consecutive days. In some embodiments, steps (b) to (d) are repeated at least once a week, for at least two weeks. In some embodiments, steps (b) to (d) are repeated at least once a week, for at least two consecutive weeks.

In some embodiments, steps (b) to (d) are repeated when desired.

In some embodiments, the disease and disorder include, but are not limited to, vaginal dryness, vulvodynia, dyspareunia, vaginismus, vaginosis, vaginal bleeding, vaginal thrush, candidiasis, vaginal fungal infection, vaginal bacterial infection, vaginal viral infection and vaginal cancer.

Vaginal dryness is prevalent among women of all ages, but is particularly common during and after the menopause. In addition, chemotherapy and/or anti-hormonal therapy related to premature menopause which is associated with vaginal dryness (Edwards and Panay, Climacteric, 2015, DOI: 10.3109/13697137.2015.1124259).

Furthermore, in the vagina, a multispecies microbiota, usually associates with bacterial vaginosis, occurs as a dense biofilm, while a lactobacilli-dominant microbiota is sparsely distributed on the vaginal epithelium (Edwards and Panay, ibid). Estrogen stimulates the proliferation of lactobacilli, reduces pH, and prevents vaginal colonization of Enterobacteriaceae, which are the main pathogens of the urinary tract. In postmenopausal women, a reduction in estrogen leads to declining lactobacilli numbers and a subsequent rise in vaginal pH, which provides favorable conditions for colonization of the vagina with Enterobacteriaceae from the rectum.

One of a highly debilitating, vaginal disorder is dyspareunia. Dyspareunia is characterized by difficult or painful sexual intercourse, and it is caused by various medical and psychological problems including vaginal stenosis and atrophy associated with aging or hormonal changes, radiation, transgender surgery, pelvic surgery or congenital abnormalities.

Vulvodynia is a vaginal disorder involving a number of syndromes, including vulvar vestibulitis, characterized by pain in the vulvar area. Vulvodynia may also affects the ability of a woman to have sexual intercourse.

Vaginismus induces involuntary spasm of the pelvic muscles surrounding the outer third of the vagina, and interferes with a woman’s ability to have a sexual intercourse. Women suffering from vaginismus are sometimes even unable to undergo a routine gynecological examination. Typical treatments of vaginismus include, for example, psychological therapy and the use of a dilator to progressively release tension and muscle spasms of the contracted muscles of the vagina. Although often effective, these treatments cause high levels of anxiety.

In some embodiments, the at least two active agents, include, but are not limited to, antifungal agents such as nystatin, butoconazole, miconazole, fenticonazole, clotrimazole, tioconazole, terconazole, econazole, ketoconazole, sulconazole, candicidin, and the like; antibacterial agents such as clindamycin, tetracycline, amoxicillin, ampicillin, erythromycin, doxycycline, lumefloxacin, norfloxacin, afloxam, ciproflaxin, azitromycin, cefltoxine, and the like; antiviral agents such as acyclovir, femciclovir, valacyclovir, AZT, and the like; antiparasitic agents such as tinidazole, miconazole, metronidazole, secnidazole, and the like; steroids such as estradiol, progesterone, diethylstilbestrol, and the like; analgesic agents such as benzocaine, tetracaine, procaine, antipyrine, and the like; anti- itch agents such as capsaicin and its derivatives, e.g., nonivamide, corticosteroids, e.g., hydrocortisone and flurandrenolide, and herbal agents such as chamomile, tea tree oil, calendula, and the like; and anti-dryness or lubricating agents such as hyaluronic acid, hyaluronic salt, vitamin E, an estrogen, an aglycon isoflavone, collagen, plant hormones such as jasmonic acid and gibberellic acid, and a retinoid or carotenoid (e.g. vitamin A). Each possibility represents a separate embodiment of the invention.

There is provided, in accordance with some embodiments, a process for preparing the multi-layered intravaginal device, disclosed herein, the process comprising the steps of:
(a) mixing water and agar, thereby obtaining an agar/water mixture;
(b) adding to the agar/water mixture an aqueous hydrogel composition comprising a plurality of naturally occurring polysaccharide and at least one cross-linking agent, thereby obtaining a hydrogel mixture;
(c) heating the hydrogel mixture to a temperature within the range of 80° C. to 100° C. while mixing;
(d) pouring the hydrogel mixture into a mold;
(e) curing the hydrogel mixture to obtain a hydrogel layer corresponding to the shape of said layer within a multi-layered intravaginal device; and
(f) adhering the hydrogel layer to an additional hydrogel layer, thereby obtaining a multilayer-layered intravaginal device having two hydrogel layers.

In some embodiments, the method further comprising preparing an additional layer and adhering the additional layer to the multilayer-layered intravaginal device having two layers, thereby obtaining a multilayer-layered intravaginal device having three layers.

The term ‘mold’ as used herein refers to a structure, typically a hollow structure having the shape/outlines of an elongated intravaginal device, or parts (layers) thereof.

In some embodiments, the agar concentration in the agar water mixture is within the range of 0.1 to 3% w/w.

The term “agar” as used herein refers to a hydrophilic colloid extracted from certain seaweeds of the Rhodophyceae class. It is insoluble in cold water but soluble in boiling water. A solution containing 1.5% w/w agar is clear/transparent, and when cooled to 34-43° C. it forms a firm gel which melt/liquidize in a temperate of, or above, 85° C. This property lends a suitable balance between easy melting and good gel stability at relatively high temperatures. Agar consists of a mixture of polysaccharides whose basic monomer is galactose. These polysaccharides can be sulphated in very variable degrees but to a lesser degree than in carrageenan. The two polysaccharides which form agar are agarose and agaropectin, with agarose making up about 70% of the agar. Agarose is a linear polymer, made up of repeating units of agarobiose, a disaccharide made up of D-galactose and 3,6-anhydro-L-galactopyranose. Agaropectin is a heterogeneous mixture of smaller molecules that occur in lesser amounts, and is made up of alternating units of D-galactose and L-galactose heavily modified with acidic side-groups, such as sulfate and pyruvate.

By ‘obtain a hydrogel layer corresponding to the shape of said layer within a multi-layered intravaginal device’ it is meant that each hydrogel layer prepared by the aforementioned process corresponds to a layer within an intravaginal device, having a specific location and shape within the device. Thus, the mold in which the hydrogel layer is set, has the shape corresponding to the shape and location of said layer in the intravaginal device which is formed from the plurality of layers.

In some embodiments, the aqueous hydrogel composition added in step (b) includes at least one additional component selected from the group consisting of: at least one preservative, at least one stabilizer, at least one co-solvent, at least one pH modifier and at least one pharmaceutically active ingredient.

The term ‘aqueous hydrogel composition’ refers to a composition comprising water as the main solvent.

In some embodiments, heating the hydrogel mixture to a temperature within the range of 80° C. to 100° C. while mixing further includes maintaining the hydrogel mixture in said temperature for at least 15 minutes while mixing.

In some embodiments, said maintaining the hydrogel mixture in said temperature is carried out for at least 20 minutes, while mixing. In some embodiments, said maintaining the hydrogel mixture in said temperature is carried out for at least 30 minutes, while mixing. In some embodiments, said maintaining the hydrogel mixture in said temperature is carried out for at least 40 minutes, while mixing. In some embodiments, said maintaining the hydrogel mixture in said temperature is carried out for at least 45 minutes, while mixing. In some embodiments, said maintaining the hydrogel mixture in said temperature is carried out for at least 50 minutes, while mixing. In some embodiments, said maintaining the hydrogel mixture in said temperature is carried out for at least 60 minutes, while mixing. In some embodiments, said maintaining the hydrogel mixture in said temperature is carried out for up to 3 hours, while mixing. In some embodiments, said maintaining the hydrogel mixture in said temperature is carried out for up to 2.5 hours, while mixing. In some embodiments, said maintaining the hydrogel mixture in said temperature is carried out for up to 2 hours, while mixing. In some embodiments, said maintaining the hydrogel mixture in said temperature is carried out for up to 1 hours, while mixing.

In some embodiments, said adhering the hydrogel layer to an additional hydrogel layer comprises preparing the additional layer according to steps (a) to (c) and pouring said additional layer onto the hydrogel layer prepared according to steps (a) to (e), namely, pouring the additional layer onto the cured hydrogel layer. It is to be understood that preparing the additional layer according to steps (a) to (c) refers to preparing an uncured hydrogel layer, or preparing a hydrogel layer in a liquid form (pre-cured), and pouring said additional uncured hydrogel layer onto the cured hydrogel layer. In some embodiments, said adhering the hydrogel layer to an additional hydrogel layer comprises preparing a liquid (pre-cured or uncured) form of the additional hydrogel layer and pouring said additional hydrogel layer onto the hydrogel layer.

In some embodiments, said pouring comprises pouring said additional layer into the hydrogel layer prepared according to steps (a) to (e), wherein said hydrogel layer is hollow and is having an opening, such that the additional layer is poured into said opening.

In some embodiments, the hollow hydrogel layer has the outlines of the intravaginal device.

In some embodiments, said adhering the hydrogel layer to an additional hydrogel layer comprises wrapping the hydrogel layer with the additional hydrogel layer. In some embodiments, the wrapping is performed compression. In some embodiments, the hydrogel layer is a core layer having the elongated three-dimensional shape of the intravaginal device, as shown for example in FIGS. 1A, 2A, 2C and 3A.

In some embodiments, said adhering the hydrogel layer to an additional hydrogel layer comprises pouring the additional layer into the mold, prior to step (e), thereby obtaining a hydrogel layer corresponding to the shape of said layer within a multi-layered intravaginal device, and letting the hydrogel layer and the additional layer cure, thereby forming the multilayer-layered intravaginal device. Thus, said adhering comprising pouring an additional hydrogel layer in a liquid form to a hydrogel layer in a liquid form, within a mold, wherein is the mold is configured to accommodate said liquid layers.

In some embodiments, said adhering the hydrogel layer to an additional hydrogel layer comprises preparing the additional layer according to steps (a) to (c); immersing the hydrogel layer obtained following steps (a) to (e) within the additional layer; and curing the additional layer, thereby obtaining a hydrogel layer corresponding to the shape of said layer within a multi-layered intravaginal device, and letting the hydrogel layer and the additional layer to cure, thereby forming the multilayer-layered intravaginal device. In some embodiments, said curing comprises pressing the hydrogel layer and the additional hydrogel layer together.

In some embodiments, the process for preparing the multi-layered intravaginal device further comprises prior to step (f) the step of mixing water and agar, thereby obtaining an agar/water adhesive layer; spreading the agar/water adhesive layer on the hydrogel layer formed in step (e); spreading the agar/water adhesive mixture on a surface of said hydrogel layer; adhering the additional layer by contacting a surface thereof with the surface of said hydrogel layer, thereby obtaining a multilayer-layered intravaginal device.

In some embodiments, said curing is at room temperature. In some embodiments, said curing is carried out 8 to 48 hours.

There is provided, in accordance with some embodiments, a process for preparing the multi-layered intravaginal device, disclosed herein, the process comprising the steps of:
(a) mixing water and agar, thereby obtaining an agar/water mixture;
(b) adding to the agar/water mixture an aqueous hydrogel composition comprising a plurality of naturally occurring polysaccharide and at least one cross-linking agent, thereby obtaining a hydrogel mixture;
(c) heating the hydrogel mixture to a temperature within the range of 80° C. to 100° C. while mixing;
(d) pouring the hydrogel mixture into a mold;
(e) curing the hydrogel mixture to obtain a cured hydrogel layer corresponding to the shape of said layer within a multi-layered intravaginal device;
(e′) repeating steps (a) to (c) thereby obtaining an additional hydrogel layer in a liquid form; and
(f) adhering the cured hydrogel layer to the additional hydrogel layer, thereby obtaining a multilayer-layered intravaginal device having two hydrogel layers.

In some embodiments, said cured hydrogel layer is hollow and is having an opening and said adhering comprises pouring said additional layer into the cured hydrogel layer through said opening. In some embodiments, the method further comprises curing the additional hydrogel layer.

In some embodiments, said adhering comprises immersing said cured hydrogel layer into the additional layer. In some embodiments, the method further comprises curing the additional hydrogel layer.

### EXAMPLES

This subsection presents specific intravaginal delivery device, which demonstrate the certain aspects of the disclosed device, kit and method.

### Example 1 - Intravaginal Delivery Device in Α Stack Structure

An intravaginal delivery device having three segments attached to one another along the Z axis (stack), as shown in FIG. 2A, is provided, wherein each segment includes a distinct active agents. The contents of each hydrogel layer, not including the pharmaceutical active ingredients, is detailed in Table 1. The compound are given along with their use where it is further indicated which compounds must be present (‘y’ means ‘yes’) and which were added, though they are optional.

TABLE 1

Contents of hydrogel layer

| Compound | Concentration (%w/w) | Use | Must | Optional |
|---|---|---|---|---|
| Water | 91.4 | Solvent | y | |
| Glycerol | 5 | Co-solvent | | y |
| Tri sodium citrate | 0.3 | Stabilizer | | y |
| Sodium benzoate | 0.05 | Preservative | | y |
| Potassium sorbate | 0.1 | Preservative | | y |
| Gellan gum low acyl | 1 | Hydrogel texture | y | |
| Carrageenan and locust | 0.5 | Hydrogel texture | y | |
| bean gum mixture | | | | |
| Gellan gum high acyl | 1 | Hydrogel texture | y | |
| KCl | 0.25 | Cross-linking agent | y | |
| CaCl₂ | 0.25 | Cross-linking agent | y | |
| Citric acid | 0.15 | pH modifier | y | |

The device was configured to address vaginal wellness and counter indications, during menopause, as reflected by the choice of agent in each distinct location/layer, along the device. The device included the following layers:
1) Top layer (layer 230) which is the first layer entering the vagina and coming in contact with the vaginal opening, includes lidocaine. Thus, the top layer includes an anesthetic, for pain relief. This layer may also cause drying and thinning of epithelium, a side effect induced by lidocaine.
2) Middle layer (layer 220) which is in contact with the second third of vaginal canal when the entire device enters the vagina, includes hyaluronic acid, for the purpose of lubrication and hydration of the internal tissue, namely, of the vaginal walls.
3) Bottom layer (layer 210) includes a composition comprising zinc as the active agent, which induces anti-inflammatory activity.

### Example2 - Intravaginal Delivery Device in Α Concentric Structure

An intravaginal delivery device having two segments attached to one another in a concentric structure, as shown in FIG. 1A, is provided, wherein each segment includes a distinct active agents. The contents of each hydrogel layer, not including the pharmaceutical active ingredients, is detailed in Table 2. The compound are given along with their use where it is further indicated which compounds must be present (‘y’ means ‘yes’) and which were added, though they are optional.

TABLE 2

Contents of hydrogel layer

| Compound | Concentration (%w/w) | Use | Must | Optional |
|---|---|---|---|---|
| Water | 89.9 | Solvent | y | |
| KCl | 0.3 | Stabilizer | | y |
| Sodium benzoate | 0.2 | Preservative | | y |
| Potassium sorbate | 0.2 | Preservative | | y |
| Gellan gum low acyl | 1 | Polysaccharide | y | |
| Glycerol | 5 | Co-solvent | | y |
| Carrageenan and locust | 1.5 | Polysaccharide | y | |
| bean gum mixture | | | | |
| Gellan gum high acyl | 1.2 | Polysaccharide | y | |
| NaCl | 0.5 | Cross-linking agent | y | |
| Lactic acid | 0.2 | pH modifier | | y |

The device was configured to address sequential delivery of the active agents for providing an extended delivery effect. The device included the following layers:
1) An external layer (layer 110) which includes lactic acid as the active agent, configured to balance vaginal pH to normal values of 3.8-4.
2) An internal layer (layer 120) which includes aloe vera as the active agent, for the purpose of hydration and for soothing (reducing itching effect which may be induced by the acid) following pH balancing. The aloe vera in the internal layer diffuses through the external layer and reaches the vaginal walls.

### Example 3 - Intravaginal Delivery Device Containing Α Hybrid Layer

An intravaginal delivery device having a hybrid hydrogel layer, including a hydrogel layer having non-hydrogel particles is provided. The contents of the hydrogel layer, is as detailed in Table 1 or in Table 2, wherein the hybrid hydrogel layer comprises:
1) A hydrogel layer; and
2) A plurality of non-hydrogel particles, the non-hydrogel particles are embedded within the hydrogel layer, wherein the non-hydrogel particles are poly(vinyl alcohol) (PVA) microcapsules encapsulating chamomile extract.

The intravaginal delivery device is intended for treating vaginal atrophy in postmenopausal women.

### Example 4 - A Process for Preparing Multi-layered Intravaginal Delivery Device

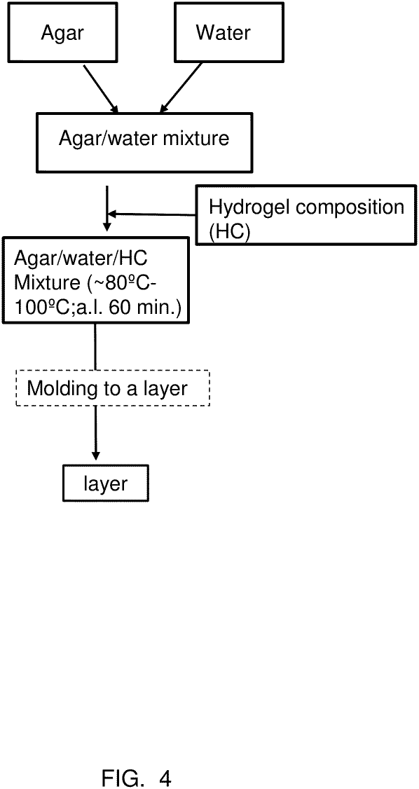

In this example, a process for making a triple layered intravaginal device is described. Agar and water were mixed, for about 10 min. (or until full transparency was observed) wherein agar concentration was 0.8 ± 0.1% w/w. The components of the hydrogel composition were added to the continuously mixed agar/water mixture, the mixture was heated, at a rate of 10° C./min. - 15° C./min., to a temperature within the range of 80° C. to 100° C. and maintained mixed under this temperature for at least 60 minutes. The details of the hydrogel composition are provided in Table 3, below. Next, the mixture is molded, to the desired shape, within a mold having the outlines of the desired shape, thereby resulting with one layer of the multi-layered intravaginal deliver device. The outlines of this process are shown in FIG. 4.

TABLE 3

Hydrogel composition

| Ingredient | Representative materials | Amount (%w/w) |
|---|---|---|
| Aquas solvent | water | at least 90% |
| Polysaccharide | Collogen, gum arabic, guar gum, konjac gum, pectin, xanthan gum, gellan gum, cellulose derivatives, chitin, chitosan, alginates | 1 - 5% |
| Preservative | e.g. Potassium sorbate, Sodium benzoate | 0.05 - 1% |
| Co-solvent | e.g. Glycerol, PEG’s | 1- 5% |
| Ion strengthening agent/cross linking agent/stabilizer | e.g. NaCl, KCl, Calcium lactate, CaCl₂ | 0.2 - 2% |
| pH modifying agent | e.g. Citric acid, Lactic acid | 0.1 - 0.3% |
| Active ingredient | e.g. Aloe vera, Hyaluronic salt, Vitamin C, Niacin amide, Vitamin E, Cannabinoids, botanical extracts | 0.5 - 10% |
| Surfactant | e.g. polysorbate | 0.1 - 2% |

Three layers where prepared, and the layers were adhered to one another, thereby forming the intravaginal delivery device. Adhering the layer may be achieved by initially preparing a first layer, then, a second layer prior to being set, namely, in a liquid phase may be directly poured and then cured to form a cured molded layer on top of the first (already molded/set/cured) layer to obtain double-layered structure. Typically, when using this step, the already set layer is within a mold that has room for receiving at least one more layer. Thus, when pouring the second layer on top of the first layer, the second layered is poured into a mold containing the molded/set first layer, and having sufficient room for receiving the second layer, and allowing it to set/mold.

Alternatively, two layers in a liquid form (prior to being set) may be compressed against one another under pressure, to obtain the double-layered structure.

Each of the aforementioned adhering step(s) can be repeated until obtaining the desired number of layers, forming the multi-layered intravaginal delivery device.

Under certain circumstances, two set layers may be adhered to one another, by spreading an adhesive layer on at least one surface of one layer which faces the layer (other layer) to which the one layered is going to be adhered to. The adhesive layer may be a layer that includes an agar/water mixture.

Each multilayer structure is left to set/cure at room temperature for 8 to 48 hours (e.g. overnight).

Any polysaccharide can be used for forming each layer, including, but not limited to, guar gum which is composed of galactose and mannose.

For the preparation of an elongated intravaginal delivery device having a plurality of hydrogel layers arranged concentrically, a core layer (e.g. layer 130) is initially prepared as described above, then dipped/immersed within a liquid form of the a second layer (e.g. layer 120). Alternatively, a second hollow layer is initially formed (e.g. layer 110) with an open end or a small opening, and then an internal layer is poured into the cavity of the hollow layer in a liquid form or poured into the opening with the hollow layer, thereby forming two-layered concentric structure. Another option is to initially prepare a core layer (e.g. layer 130) as described above, then wrap the core layer with another hydrogel layer, under compression. Any one of the aforementioned processes may be used for forming a multi-layered elongated intravaginal delivery device having a hydrogel layers arranged concentrically.

For the preparation of an elongated intravaginal delivery device having a plurality of hydrogel layers embedded, and dispersed, in a leading hydrogel layer, the embedded hydrogel layer is prepared either in a dry form or encapsulated, e.g. within liposomes or any other particle which is medically approved. The particles are added to a hydrogel layer in a liquid form (pre-setting) and the mixture is molded to the desired shape and left to set, as described above.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

1-28. (canceled)

29. An intravaginal delivery device comprising a plurality of hydrogel layers, wherein adjacent hydrogel layer layers are attached to one another, and wherein the layers of the plurality of hydrogel layers are different from one another by content.

30. The intravaginal delivery device of claim 29, wherein the plurality of hydrogel layers is arranged concentrically and comprises a core hydrogel layer and an external hydrogel layer.

31. The intravaginal delivery device of claim 30, further comprising at least one transitional hydrogel layer.

32. The intravaginal delivery device of claim 29, wherein the plurality of hydrogel layers is arranged in a stack.

33. The intravaginal delivery device of claim 29, wherein at least one layer of the plurality of layers is a foundational layer comprising at least one other layer dispersed therewithin.

34. The intravaginal delivery device of claim 33, wherein the at least one other layer is in the form of particles, wherein the at least one other layer comprises a plurality of distinct layers, and wherein the particles are randomly dispersed within the foundational layer.

35. The intravaginal delivery device of claim 29, wherein at least one layer of the plurality of hydrogel layers comprises a hybrid hydrogel.

36. The intravaginal delivery device of claim 29, wherein at least one layer of the plurality of hydrogel layers comprises a pharmaceutical composition comprising at least one active agent.

37. A kit comprising the intravaginal delivery device of claim 29, a case configured to enclose the intravaginal delivery device therewithin, a pH indicator, and instructions for use of the intravaginal delivery device for delivering, intravaginally, a plurality of active agents.

38. The kit of claim 37, wherein the case is a solid case and wherein the case is configured to hermetically enclose the intravaginal delivery device therewithin.

39. Use of an intravaginal delivery device according to claim 29, for the treatment of a disease or disorder.

40. A process for preparing a multi-layered intravaginal device, the process comprising the steps of:
(a) mixing water and agar, thereby obtaining an agar/water mixture;
(b) adding to the agar/water mixture an aqueous hydrogel composition comprising a plurality of naturally occurring polysaccharide and at least one cross-linking agent, thereby obtaining a hydrogel mixture;
(c) heating the hydrogel mixture to a temperature within the range of 80° C. to 100° C. while mixing;
(d) pouring the hydrogel mixture into a mold;
(e) curing the hydrogel mixture to obtain a hydrogel layer corresponding to the shape of said mold; and
(f) adhering the hydrogel layer to an additional hydrogel layer, thereby obtaining a multilayer-layered intravaginal device having two hydrogel layers.

41. The process according to claim 40, wherein the agar concentration in the agar water mixture is within the range of 0.1 to 3% w/w.

42. The process according to claim 40, wherein the aqueous hydrogel composition added in step (b) further comprises at least one additional component selected from the group consisting of: at least one preservative, at least one stabilizer, at least one cosolvent, at least one pH modifier and at least one pharmaceutically active ingredient.

43. The process according to claim 40, wherein said adhering the hydrogel layer to an additional hydrogel layer comprises preparing a liquid form of the additional hydrogel layer and pouring said additional hydrogel layer onto the hydrogel layer.

44. The process according to claim 40, wherein said adhering the hydrogel layer to an additional hydrogel layer comprises preparing a liquid form of the additional hydrogel layer; pouring said additional hydrogel layer into the hydrogel layer; and curing said additional hydrogel layer.

45. The process according to claim 40, wherein said adhering the hydrogel layer to an additional hydrogel layer comprises wrapping the hydrogel layer with the additional hydrogel layer.

46. The process according to claim 40, wherein said adhering the hydrogel layer to an additional hydrogel layer comprises preparing the additional layer according to steps (a) to (c); immersing the hydrogel layer obtained within the additional hydrogel layer; and curing the additional hydrogel layer.

47. The process according to claim 40, further comprising the step of mixing water and agar, thereby obtaining an agar/water adhesive layer; spreading the agar/water adhesive layer on the hydrogel layer prior to step (f); spreading the agar/water adhesive mixture on a surface of said hydrogel layer; and adhering the additional layer by contacting a surface thereof with the surface of said hydrogel layer.

48. The process according to claim 40, wherein said curing is carried out for 8 to 48 hours.
